(19) United States
(12) Patent Application Publication
(10) Pub. No.: US 2015/0037900 A1
(43) Pub. Date: Feb. 5, 2015

(54) Point of Care Cyanide Test Device and Kit

(71) Applicant: Cornell University, Ithaca, NJ (US)

(72) Inventor: Joseph Rella, Short Hills, NJ (US)

(21) Appl. No.: 14/516,147

(22) Filed: Oct. 16, 2014

Related U.S. Application Data
(63) Continuation of application No. PCT/US2013/037360, filed on Apr. 19, 2013.
(60) Provisional application No. 61/635,421, filed on Apr. 19, 2012.

(51) Int. Cl. G01N 33/49; G01N 27/12; G01N 31/22

(52) U.S. Cl. CPC G01N 33/4925; G01N 31/224; G01N 27/126
USPC 436/109; 422/90; 422/82.09

(57) ABSTRACT

A device, method, and test kit for rapidly detecting cyanide in a sample. The inventive device comprises a container comprising a sample chamber and a sensor chamber separated by a selectively permeable barrier. The sample chamber contains a reagent for releasing cyanide from the sample, and the sensor chamber contains a cyanide detector comprising a conductive polymer which absorbs the released cyanide, generating a change in signal. Signals can be viewed colorimetrically or transmitted to a LCD/LED panel wherein the cyanide measurement readout is displayed.

### CROSS-REFERENCE TO RELATED APPLICATIONS

This application is a continuation of PCT Application No. PCT/US2013/037360 filed Apr. 19, 2013 which claims priority to U.S. Provisional Application Ser. No. 61/635,421 filed Apr. 19, 2012, the entire contents of which are incorporated by reference herewith.

### FIELD OF THE INVENTION

This invention relates to a point of care device, method, and test kit for rapidly detecting cyanide in samples.

### BACKGROUND OF THE INVENTION

Cyanide is a very potent neurological and metabolic poison. House fires generate cyanide gas when materials commonly found in homes and other buildings combust. In addition to traumatic injuries, burns, smoke inhalation, and carbon monoxide poisoning, cyanide is commonly found (after the fact) to have been another element contributing to a patient's morbidity and mortality. In addition to this common source of poisoning, cyanide is also considered to be a weapon of mass destruction and may be used in an act of terrorism or in war.

Although there are many laboratory methods that can measure cyanide, none can provide a measurement with enough rapidity (i.e., within minutes) to make a clinical difference to a poisoned patient. Often, results are available to physicians only after 2 weeks. Current tests provide a laboratory-based assay only, meaning once blood is drawn from the patient and the appropriate lab is identified, blood must then be shipped to a remote laboratory and the final report may return days to weeks later. Several existing methods that require laborious multistep sample pre-treatment are not amenable for use in the field.

Since cyanide is a very rapidly acting toxin, it is imperative that physicians and other health care providers have available a rapid test system so that they may provide the best care possible for sick patients because this delay does not allow for an immediate answer and/or treatment for the patient who is suffering from the poisoning. Additionally, empiric treatment for presumed cyanide poisoning is inconsistently used with controversial adverse clinical effects.

Thus there remains a need for improved devices, assays or test kits for rapidly detecting the presence of cyanide in a sample obtained from a subject, providing a point of care and treatment immediately for such subject if cyanide is detected.

### SUMMARY OF THE INVENTION

The invention provides a point of care device, method, and test kit for rapidly detecting the presence of cyanide in a sample.

In certain embodiments, the invention provides a device for cyanide detection comprising: a) a sealable container comprising a sample chamber and a sensor chamber, b) a cap for sealing an open end of the container; c) a liquid impermeable and gas permeable membrane anchored to an inner surface of the container separating the sample chamber from the sensor chamber; d) a reagent inside the sample chamber that separates and releases cyanide from the sample; and e) a cyanide detector inside the sensor chamber comprising a conducting polymer that absorbs released cyanide, wherein the conducting polymer can act as a semiconductor to measure cyanide quantitatively from the test sample.

The inventive device can be used for detecting cyanide in any sample, including but not limited to, environmental fluids, or bodily fluids, such as blood, saliva, tears, and urine obtained from any animals, such as mammals including but not limited to humans, livestock including but not limited to bovine, porcine, and ovine, and companion animals including but not limited to canine and feline. In certain embodiments, the test sample is human blood.

In certain embodiments, the invention provides a device comprising a cyanide detector comprising a conducting polymer comprising aromatic cycles with or without heteroatoms present. In certain embodiments, the conducting polymer comprises aromatic cycles without heteroatoms present, such conducting polymer includes, but not limited to, polyfluorene, polyphenylene, polypyrene, polyazulene, and polynaphthalene. In other embodiments, the conducting polymer comprises aromatic cycles in which at least one nitrogen atom is present in an aromatic cycle, such conducting polymer includes, but not limited to, polypyrrole, polycarbazole, polyindole, and polyazepine. In yet other embodiments, the conducting polymer comprises aromatic cycles in which at least one nitrogen atom is outside an aromatic cycle, and such conducting polymer includes, but is not limited to, polyaniline (PANI).

In yet other embodiments, the conducting polymer comprises heteroatoms in which at least one sulfur atom is present in an aromatic cycle, such conducting polymer includes, but not limited to, polythiophene. In yet other embodiments, the conducting polymer comprises heteroatoms in which at least one sulfur atom is outside an aromatic cycle, such conducting polymer includes, but not limited to, poly(p-phenylene sulfide).

In certain embodiments, the conducting polymer comprises at least one double bond, such conducting polymer includes, but not limited to, polyacetylene. In some other embodiments, the conducting polymer is a copolymer comprising any mixture of a polymer having at least one aromatic cycle and a polymer having at least one double bond. Such conducting copolymer includes, but not limited to, poly(p-phenylene vinylene).

In certain embodiments, the conducting polymer in the cyanide detector can be formed into nanotubes or serve as a coating to carbon nanotubes or in any other form. In one embodiment, the cyanide detector in the inventive device is formed into test strip, such as the CYANTESMO test strip. In certain embodiments, the cyanide detector in the inventive device may be anchored to the bottom surface of the cap, or to a projection from the bottom surface of the cap, or the sides of the tube.

The invention further provides that the sensor chamber of the inventive device further comprises a signal processor wherein the cyanide detector is operably coupled therewith. In some embodiments, the processor in the inventive device further comprises a power or energy supply source, which may be embedded within or attached to the cap.

The invention further provides that the sensor chamber of the inventive device further comprises a display for cyanide measurement readout. In some embodiments, the display includes, but not limited to, an LCD or LED display, which may be embedded within or attached to the cap, as well.

The invention further provides that the sample chamber has an internal pressure less than atmosphere pressure for drawing a volume of the test sample into the sample chamber through a re-sealable cap on the sample chamber end of the container.

The invention provides that the sample chamber of the inventive device comprises reagents capable of separating and release cyanide from the sample. Exemplary reagents include, but are not limited to, acids. In certain embodiments, the reagents may comprise at least one acid able to denature the test sample, including but not limited to, phosphoric acid, sulfuric acid, and ascorbic acid. The reagents in the sample chamber can be in any form including, but not limited to, a liquid, gel, or solid form.

Methods for rapid cyanide detection using the device of the invention are also provided. The invention method comprises the steps of: a) placing a sample in the sample chamber, and b) reviewing the detector or display for an indication of the presence or amount of cyanide. In certain embodiments, the inventive method comprises: a) mixing the sample with at least one reagent to separate and release cyanide from the sample; b) absorbing the released cyanide by the cyanide detector comprising the conducting polymer; c) quantifying the cyanide concentration with the cyanide detector; d) processing the quantified cyanide concentration into a signal by a processor; and e) transmitting the signal to the display for cyanide measurement readout.

The invention further provides a rapid cyanide detection kit comprising the device of the invention and instructions on how to use the device. In certain embodiments, the cyanide test kit may have reagents already installed in the device or provided separately. The kit may include a phlebotomy needle and holder for obtaining a blood sample. The invention provides that the cyanide test kit is appropriate for a point of care test system for cyanide detection and subsequent treatment.

### BRIEF DESCRIPTION OF THE DRAWINGS

FIG. 1 illustrates one embodiment of the inventive device comprising a tube container, a cap, a LED display, a cyanide detector, and a liquid impermeable, gas permeable membrane.

### DETAILED DESCRIPTION OF THE INVENTION

In the following description, reference is made to the accompanying drawings that form a part hereof, and in which is shown by way of illustration specific embodiments which may be practiced. These embodiments are described in detail to enable those skilled in the art to practice the invention, and it is to be understood that other embodiments may be utilized and that logical changes may be made without departing from the scope of the present invention. The following description of example embodiments is, therefore, not to be taken in a limited sense, and the scope of the present invention is defined by the appended claims.

The Abstract is provided to comply with 37 C.F.R. §1.72(b) to allow the reader to quickly ascertain the nature and gist of the technical disclosure. The Abstract is submitted with the understanding that it will not be used to interpret or limit the scope or meaning of the claims.

The invention provides a point of care device, method, and test kit for detecting the presence of cyanide in a sample. The sample may be obtained from the environment or from a subject who is suspected of having cyanide poisoning. In certain embodiments, the invention provides a device for cyanide detection comprising: a) a sealable container comprising a sample chamber and a sensor chamber, b) a cap for sealing an open end of the container; c) a liquid impermeable and gas permeable membrane anchored to an inner surface of the container separating the sample chamber from the sensor chamber; d) a reagent inside the sample chamber capable of releasing cyanide from the sample; and e) a cyanide detector inside the sensor chamber. In certain embodiments, the cyanide detector is a conducting polymer that absorbs the released cyanide, wherein the conducting polymer acts as a semiconductor to measure cyanide quantitatively from the test sample.

The inventive device can be used for detecting cyanide in any samples, such as environmental or biological samples, including but not limited to, blood, saliva, tears, and urine obtained from any mammal including but not limited to humans, livestock including but not limited to bovine, porcine, and ovine, and companion animals including but not limited to, canine and feline. In certain embodiments, the test sample is human blood.

The container of the inventive device comprises a sample chamber with a sample inert therein and a sensor chamber. In certain embodiments, the sample chamber within the container can be a self-contained unit that is able to access standard sample draw systems, such as VACUTAINER system for blood collection (available from Becton Dickinson, Franklin Lakes, N.J.). As used herein, a container refers to an item suitable for use to contain, hold, store, and transport a biological sample. Such containers can have sealable stoppers or caps on the sample chamber side of the container configured for blood sample collection with a phlebotomy needle and holder, which are well-known in the art. The container can be, but is not limited to, a tube, bottle, jar, and any other items now known or later developed in the art that can be used for holding a biological sample. In certain embodiments, the container can be clear glass or plastic and marked, for instance, with a fill line for the desired volume of sample, e.g., blood.

In certain embodiments, the inventive device also comprises a cap for of an sealing at least one or both of an open end of the container on the sensor side or the sample side of the container. As used herein, a cap refers to an item suitable to seal or close an open end of a container. The cap may be any cap now known or later developed in the art that can be used for sealing or closing an open end of a container holding a biological sample. The cap can be made of any materials, including but not limited to, plastic, rubber, and metal, and may be colored or otherwise marked.

The bottom of the cap can be attached to any projections for certain usage. In certain embodiments where the cap is on the sensor chamber side of the container, certain elements, such as the signal processor, the energy source, and the display within the sensor chamber of the inventive device may be embedded within or attached to the cap or any projections from the cap. In certain embodiments where the cap is on the sample chamber side of the container, the cap is made of a re-sealable polymer or rubber material which can be punctured with a phlebotomy needle.

In certain embodiments, the inventive device may be packaged with the container capped with most or some of the air removed from the interior of the container. A perfect vacuum need not be created as the desired effect is negative pressure with respect to the surrounding air, in order facilitate the sample draw, as is well-known in the art.

The inventive device also comprises a liquid impermeable, gas permeable membrane anchored to the inner surface of the container, which separates the sample chamber from the sensor chamber. In certain embodiments, this membrane is located between the sample level fill line on the container and the bottom of the cap or any projections from the cap for separating the sample chamber from the sensor chamber. Any suitable liquid impermeable and gas permeable membrane, now known or later developed in the art, which allows released gaseous cyanide to pass from the sample chamber and maintains the fluid sample in the sample chamber, is encompassed by the invention.

The inventive device also comprises a cyanide detector located inside of the sensor chamber. Any suitable cyanide detection technologies now available or later developed in the art, are encompassed in the invention. Exemplary cyanide detectors and detection methods are described in Ma and Dasgupta (2010), Anal Chim Acta. 673(2): 117-125; Rella et al. (2004), J Toxico Clin Toxicol. 42(6): 897-900; Ma (2011), Anal Chem. 83(11):4319-24; Murphy et al. (2006), Clin. Chem. 52(3):458-467; and US Publication No. 2013/0005044 to Boss et al., each of which is herein incorporated by reference in its entirety.

In certain embodiments, the cyanide detector within the sensor chamber of the inventive device comprises a conducting polymer, which can be a copolymer. As used herewith, the term “conducting polymer” refers to any polymers that are able to conduct electricity and acts as a semiconductor to measure cyanide quantitatively from the sample. Conducting polymers are well-known in the art, and the invention is not limited in scope to any particular conducting polymers being used in the cyanide detector. When a conducting polymer is used, the released hydrogen cyanide (HCN) protonates or dopes the conducting polymer proportional to the concentration of the HCN. The conductance of the copolymer varies directly with the degree of HCN doping.

In certain embodiments, the main chain of the conducting polymer comprises aromatic cycles. In some embodiments, the aromatic cycles have no heteroatoms present. Exemplary conducting polymer in this group includes but not limited to, polyfluorene, polyphenylene, polypyrene, polyazulene, and/or polynaphthalene. In other embodiments, the main chain of the conducting polymer can comprise heteroatoms, in which at least one nitrogen atom is present in an aromatic cycle. Exemplary conducting polymer in this group includes but not limited to, polypyrrole, polycarbazole, polyindole, and/or polyazepine. In other embodiments, the conducting polymer can comprise heteroatoms, in which at least one nitrogen atom can be outside an aromatic cycle. Exemplary conducting polymer in this group is polyaniline (PANI). In some embodiments, the main chain of the conducting polymer comprises heteroatoms, in which at least one sulfur atom is present in an aromatic cycle. An exemplary conducting polymer in this group is polythiophene. In other embodiments, the main chain of the conducting polymer comprises heteroatoms, in which at least one sulfur atom can be outside an aromatic cycle. An exemplary conducting polymer in this group is poly(p-phenylene sulfide).

In certain embodiments, the main chain of the conducting polymer can comprise at least one double bond. Exemplary conducting polymer in this group is polyacetylene. In yet other embodiments, the main chain of the conducting polymner used in the cyanide detector comprises any mixture of any conducting polymers, now known or later development in the art. Such conducting copolymer may comprise at least one aromatic cycle and at least one double bond. Exemplary conducting copolymer in this group is poly(p-phenylene vinylene).

In certain embodiments, the cyanide detector of the invention can be present in any useful form. For example, a conducting polymer may be formed into nanotubes or serve as a coating to carbon nanotubes or in any other form. In one embodiment, the cyanide detector in the inventive device is formed into test strip, such as the CYANTESMO test strip or paper (available from Machery-Nagel GmbH & Co., Duren, Germany). In certain embodiments, the cyanide detector may be anchored directly to the inside of the container, to the bottom surface of the sensor cap, or a projection from the bottom surface of the sensor cap, or may be anchored to the sides of the container.

In certain embodiments, the cyanide detector within the inventive device is operably coupled to a signal processor, which may further comprise a power or energy supply. When a conducting polymer is used within the cyanide detector, a small current is supplied within the device and conducted through the conducting polymer. The change in signal is then read by the signal processor which in turn transmits a signal to the display, such as an LCD or LED display for cyanide measurement readout, thus providing the patient or healthcare provider with a cyanide concentration measurement in the sample. In certain embodiments, the signal processor, the energy source for the processor, and the LCD or LED display may be embedded within or attached to the cap.

The sample chamber within the inventive device comprises reagents that are capable of separating and releasing cyanide from the test sample. In certain embodiments, the reagent includes at least one acid that is able to denature the test sample and release gaseous cyanide from the test sample. Exemplary acids include but are not limited to phosphoric acid, sulfuric acid, and ascorbic acid. The reagent may be pre-installed in the sample chamber within the device, and can be provided in any form including but not limited to, liquid, gel, solid, or any other forms known in the art. In certain embodiment, the reagent is in a solid form as pellet.

The sample chamber within the inventive device can have an internal pressure less than atmosphere pressure for drawing a volume of the test sample into the sample chamber. In certain embodiments, the internal pressure is generated by a vacuum embedded inside the sample chamber.

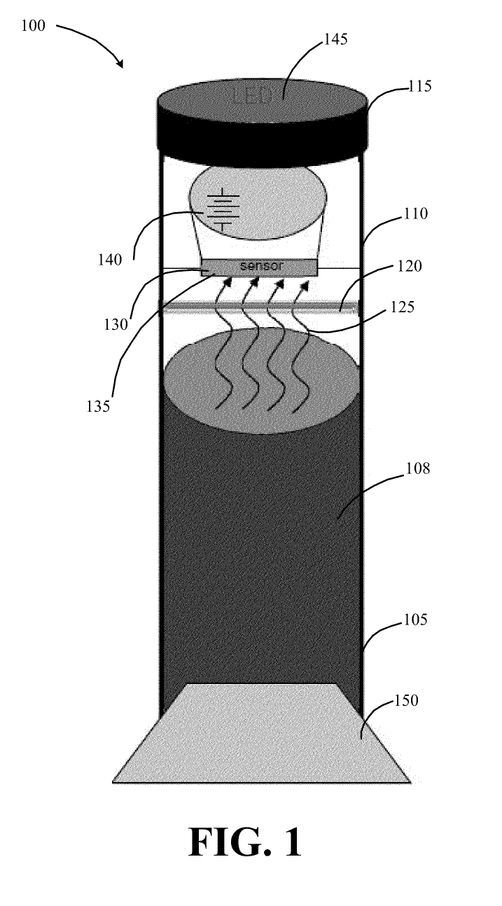

A non-limiting embodiment of the inventive device is depicted in FIG. 1, which is referenced herein. The inventive device depicted in FIG. 1 comprises a tube container (100) comprising a sample chamber (105) and a sensor chamber (110). A cap (115) for sealing the end of the container on the sensor side is provided. Another cap (150) is provided on the opposite sample collection side. A liquid impermeable and gas permeable membrane (120) is anchored to an inner surface of the container separating the sample chamber (105) from the sensor chamber (110). An acid reagent (not shown) is included inside the sample chamber (105) with blood (108) capable of separating and releasing cyanide (125) from the sample. A cyanide detector (130) is inside the sensor chamber (110) comprising a conducting polymer (135) that absorbs the released gaseous cyanide (125).

The invention further provides a method for rapid cyanide detection using the device of the invention. The inventive method comprises the steps of: a) placing a sample in the sample chamber, and b) reviewing the detector color change or electronic display for an indication of cyanide detection. In certain embodiments, the inventive method comprises: a) mixing the sample with at least one reagent to separate and release cyanide from the sample; b) absorbing the released cyanide by the cyanide detector comprising the conducting polymer; c) quantifying cyanide concentration with the cyanide detector; d) processing the quantified cyanide concentration into a signal by a processor; and e) transmitting the signal to the display for cyanide measurement readout.

In one embodiment, the inventive device depicted in FIG. 1 is used for detecting cyanide concentration from a human blood sample. Blood is drawn up into the sample chamber (105) through a resealable membrane stopper cap (150) to a volume of about 3 mL. The blood sample is then mixed with an acid inside the sample chamber (105), wherein the blood is denatured and the bound cyanide is released. The hydrogen cyanide (HCN) gas (125) is released, passing through the liquid impeameable and gas permeable membrane (120) into the sensor chamber (110) wherein the conducting polymer (135) of the cyanide detector (130) absorbs the released HCN gas, resulting in the change in signal, which is read by a signal processor (140) which is operably coupled with the cyanide detector (130) in the sensor chamber (110). The processor (140) then transmits the signal to the display (145) wherein the cyanide measurement readout is displayed.

Depending on the detection polymer used, the results may be apparent through colorimetric change, or a display reading, within several minutes or hours.

The invention further provides a rapid cyanide detection kit comprising the device of the invention, a needle and hub holder for collecting blood in the device, and instructions on how to use the device. In certain embodiments, the cyanide test kit may have reagents already installed in the device. The invention provides that the cyanide test kit is appropriate for a point of care test system for cyanide detection and subsequent treatment. For instance, at the bedside, a patient's blood is drawn into the system, similar to other standard blood tubes used for routine blood sampling (e.g., a VACUTAINER), where it uniquely mixes with a reagent to release gaseous cyanide. The inventive cyanide detector within the inventive device then measures the cyanide concentration released from the sample blood and reports that number via a numerical readout to the healthcare provider, who can then make an informed decision of whether or not to treat the patient further such as with the use an antidote.

### EXAMPLE

The present description is further illustrated by the following example, which should not be construed as limiting in any way. The contents of all cited references (including literature references, issued patents, or published patent applications as cited throughout this application) are hereby expressly incorporated by reference.

This example illustrates the inventive device and method of use thereof, for detecting cyanide in blood. The ability to detect cyanide in blood is based on previous work, where the presence of cyanide was determined spectrophotomically. It was previously demonstrated that CYANTESMO test strips, used by water treatment facilities and medical examiners, accurately and rapidly detected, in a semi-quantifiable manner, concentrations of cyanide greater than 1 mg/L in water. CYANTESMO test strips or papers are known to allow the quick and easy detection of hydrocyanic acid (HCN) and cyanides in aqueous solutions and extracts.

This example demonstrates an effective method for rapid detection of clinically important concentrations of cyanide in blood using CYANTESMO test strips as a cyanide detector in the inventive device. Varying standardized dilutions of KCN ranging from 0.5 mg/L to 30 mg/L were added to pooled, discarded blood that had been warmed to 37° C. in a water bath for 30 minutes. Test samples were then acidified with 100 μL of sulphuric acid in a closed system under a ventilation hood at room temperature. CYANTESMO registered test strips were placed into the test tubes just above the fluid level where liberated HCN gas interacted with the test strip to effect a color change. Color changes were compared to negative controls and to each other. The test strips demonstrated an incrementally increasing deep blue color change over a progressively longer portion of the test strip in less than 5 minutes for each concentration of KCN including 3, 10, and 30 mg/L. The concentrations of 0.5, and 1 mg/L did not demonstrate any color change in less than 2 hours. The CYANTESMO test strips accurately and rapidly detected, in a semi-quantifiable manner, concentrations of CN greater than 1 mg/L contained in each test sample of human blood.

1. A device for cyanide detection comprising:
a) a sealable container comprising a sample chamber and a sensor chamber,
b) a cap for sealing an open end of the container;
c) a liquid impermeable and gas permeable membrane anchored to an inner surface of the container separating the sample chamber from the sensor chamber;
d) a reagent inside the sample chamber that releases cyanide from the sample; and
e) a cyanide detector inside the sensor chamber comprising a conducting polymer that absorbs released cyanide.

2. The device of claim 1, wherein said conducting polymer comprises aromatic cycles having no heteroatoms present, said conducting polymer selected from the group consisting of polyfluorene, polyphenylene, polypyrene, polyazulene, and polynaphthalene.

3. The device of claim 1, wherein said conducting polymer comprises heteroatoms in which at least one nitrogen atom is present in or outside an aromatic cycle, said conducting polymer selected from the group consisting of polypyrrole, polycarbazole, polyindole, polyazepine, and polyaniline.

4. The device of claim 1, wherein said conducting polymer comprises heteroatoms in which at least one sulfur atom is present in or outside an aromatic cycle, and said conducting polymer is polythiophene or poly(p-phenylene sulfide).

5. The device of claim 1, wherein said conducting polymer comprises at least one double bond, and said conducting polymer comprises polyacetylene.

6. The device of claim 1, wherein said conducting polymer comprises any mixture of a polymer having at least one aromatic cycle and a polymer having at least one double bond, and said conducting copolymer comprises poly(p-phenylene vinylene).

7. The device of claim 1, wherein said conducting polymer is formed into nanotubes or serves as a coating to carbon nanotubes.

8. The device of claim 1, wherein said cyanide detector is formed into a test strip.

9. The device of claim 1, wherein said cyanide detector is anchored to the bottom surface of the cap, a projection from the bottom surface of the cap, or the sides of the tube.

10. The device of claim 1, wherein said sensor chamber further comprises a signal processor wherein the cyanide detector is operably coupled therewith.

11. The device of claim 10, wherein said processor further comprises an energy supply source.

12. The device of claim 1, wherein said sensor chamber further comprises an LCD or LED display for cyanide measurement readout.

13. The device of claim 1, wherein the cap seals the sample chamber.

14. The device of claim 13, wherein the cap is a re-sealable stopoper that can be penetrated with a phlebotomy needle.

15. The device of claim 1, wherein the container has an internal pressure less than atmospheric pressure for drawing a volume of sample into the sample chamber.

16. The device of claim 1, wherein said reagent is an acid in liquid, gel, or solid form.

17. The device of claim 1, wherein said sample is a biological sample selected from the group consisting of blood, saliva, tears, and urine.

18. A method for rapid cyanide detection using the device of claim 1, comprising:
a) placing a sample in the sample chamber, and
b) reviewing the detector for an indication of the presence of cyanide.

19. The method of claim 18, further comprising:
a) mixing the sample with at least one reagent to separate and release cyanide from the sample;
b) absorbing the released cyanide by the cyanide detector;
c) quantifying cyanide concentration with the cyanide detector;
d) processing the quantified cyanide concentration into a signal by a processor; and
e) transmitting the signal to the display for cyanide measurement readout.

20. A rapid cyanide detecting kit comprising the device of claim 1, a phlebotomy needle for blood collection, and instructions for using the device.
